(19) United States
(12) Patent Application Publication
(10) Pub. No.: US 2004/0058401 A1
(43) Pub. Date: Mar. 25, 2004

(54) Method for detecting rare event

(76) Inventors: Blaise Bossy, Carlsbad, CA (US); Robert t. Ellis, Dana Point, CA (US); Kenneth D. Bauer, San Clemente, CA (US)

Correspondence Address: FISH & RICHARDSON, PC, 12390 EL CAMINO REAL, SAN DIEGO, CA, 92130-2081, US

(21) Appl. No.: 10/618,577

(22) Filed: Jul. 11, 2003

Related U.S. Application Data
(63) Continuation-in-part of application No. 09/619,033, filed on Jul. 19, 2000, now abandoned.
(63) Continuation-in-part of application No. 10/081,714, filed on Feb. 20, 2002, now Pat. No. 6,631,203.
Application No. 10/081,714 is a continuation of application No. 09/344,308, filed on Jun. 24, 1999, now Pat. No. 6,418,236.
(60) Provisional application No. 60/144,529, filed on Jul. 19, 1999.
(60) Provisional application No. 60/129,384, filed on Apr. 13, 1999.

(51) Int. Cl. G01N 33/574; G06K 9/00

(52) U.S. Cl. 435/7.23; 382/128

(57) ABSTRACT

Provided are methods, compositions and kits for efficiently and accurately identifying rare events in a biological sample.

### CROSS REFERENCE TO RELATED APPLICATIONS

This application is a continuation-in-part and claims priority under 35 U.S.C. §120 to U.S. application Ser. No. 09/619,033 filed Jul. 19, 2000, which application claims priority under 35 U.S.C. §119 to U.S. Provisional Application Serial No. 60/144,529, filed Jul. 19, 1999. This application is also a continuation-in-part of U.S. application Ser. No. 10/081,714 filed Feb. 20, 2002, which is a continuation (and claims the benefit of priority under 35 U.S.C. §120) of U.S. application Ser. No. 09/344,308, filed Jun. 24, 1999, which claims the benefit of priority under 35 U.S.C. §119 of U.S. Provisional Application Serial No. 60/129,384, filed Apr. 13, 1999, the disclosures of the foregoing applications are incorporated herein by reference.

### FIELD OF THE INVENTION

The invention generally relates to cell proliferative disorders and more particularly to enriching cells having a cell proliferative disorder in a biological sample and identifying such rare events.

### BACKGROUND

Cell proliferative disorders can be characterized by a number of cellular changes, including expression of growth factors, growth factor receptors, adhesion molecules, and other cellular determinants, which are readily identifiable to those of skill in the art.

The detection of cell proliferative disorders is important in detecting, diagnosing and treating neoplasms, and cancers. The detection limits of many assays are not sufficient to detect cells having proliferative disorders because the number of cells present in a sample are too few to provide a detectable signal.

Accordingly, there is a desire to increase the signal in order to adequately determine the presence or type of a cell proliferative disorder in a subject.

### SUMMARY

The invention provides methods that are capable of efficiently and more accurately locate and identify rare events in a biological sample.

The invention provide a method for identifying rare events in a biological sample, comprising: obtaining a source of cells; contacting the source with a binding agent specific for a cell specific marker associated with a rare event wherein the binding agent is bound to a magnetic bead and wherein the binding agent binds to cells in the source expressing the cell specific marker; separating cells bound by the binding agent from the source thereby obtaining a sub-population of cells enriched for the cell specific marker associated with the rare event; placing the enriched sample on a substrate; automatically scanning the substrate at a plurality of coordinates; automatically obtaining a plurality of images at locations on the substrate that comprise the enriched sample; and processing the plurality of image to identify the rare event. In one aspect the binding agent is an antibody.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a perspective view of an apparatus for automated cell image analysis.

FIG. 2 is a block diagram of the apparatus shown in FIG. 1.

FIG. 3 is a plan view of the apparatus of FIG. 1 having the housing removed.

FIG. 4 is a side view of a microscope subsystem of the apparatus of FIG. 1.

FIG. 5 shows a slide carrier. FIG. 5a is a top view of a slide carrier for use levity the apparatus of FIG. 1. FIG. 5b is a bottom view of the slide carrier of FIG. 5a.

FIG. 6 shows views of an automated slide handling subsystem. FIG. 6a is a top view of an automated slide handling subsystem of the apparatus of FIG. 1. FIG. 6b is a partial cross-sectional view of the automated slide handling subsystem of FIG. 6a taken on line A-A .

FIGS. 7a-7d illustrate the output operation of the automated slide handling subsystem.

FIG. 8 is a flow diagram of the procedure for automatically determining a scan area.

FIG. 9 is a block diagram of the microscope controller of FIG. 2

FIG. 10 shows a method of histological reconstruction.

Like reference symbols in the various drawings indicate like elements.

### DETAILED DESCRIPTION

Detection of rare events such as rare cells in a sample is of importance in diagnostics and research. Techniques currently used to identify rare events utilize techniques based in positive or negative selection to enrich a sample for a specific event type (e.g., cell type). Although such methods are advantageous such techniques are inherently limited. The invention provides methods for enrichment as well as identification of rare event types that are better than positive or negative selection alone. The methods of the invention can be used in the diagnosis of mutation in cells and tissues as well as in research.

In addition, a problem with existing automated systems is the continued need for operator input to initially locate cell objects for analysis. Such continued dependence on manual input can lead to errors including objects of interest being missed. These errors can be critical especially in assays for so-called rare events, e.g., finding one stained cell in a cell population of one million normal cells. Additionally, manual methods can be extremely time consuming and can require a high degree of training to properly identify or quantify cells. The associated manual labor leads to a high cost for these procedures in addition to the potential errors that can arise from long, tedious manual examinations. A need exists, therefore, for an improved system, which can quickly and accurately scan large amounts of biological material on a slide.

Mutation is the process whereby changes occur in the quantity or structure of the genetic material of an organism. Mutations are permanent alterations in the genetic material that may lead to changes in phenotype. Mutations can involve modifications of the nucleotide sequence of a single gene, blocks of genes or whole chromosomes. Changes in single genes may be the consequence of point mutations, which involve the removal, addition, or substitution of a single nucleotide base within a DNA sequence, or they can be the consequence of changes involving the insertion or deletion of large number of nucleotides.

Modifications of whole chromosomes include both changes in number or structural changes involving chromosome abnormalities. Numerical chromosome mutations can involve multiples of the complete karyotype, termed “polyploidy,” or they may involve deviations from the normal number of chromosomes, termed “aneuploidy.”

Mutations can arise spontaneously as a result of events such as errors in the fidelity of DNA replication or the movement of transposable genetic elements within genomes. They are also induced following exposure to chemical or physical mutagens. Such mutation-inducing agents include ionizing radiations, ultraviolet light and a diverse range of chemicals such as the alkylating agents, and polycyclic aromatic hydrocarbons, all of which are capable of interacting either directly or indirectly (generally following some metabolic biotransformations) with nucleic acids. The DNA lesions induced by such environmental agents may lead to modifications of the base sequence when the affected DNA is replicated or repaired and thus to a mutation.

An increasing body of evidence implicates somatic mutations as causally important in the induction of human cancers. These somatic mutations may accumulate in the genomes of previously normal cells, some of which may then demonstrate the phenotypes associated with malignant growth. Such oncogenic mutations may include a number of different types of alterations in DNA structure, including deletions, translocations, and single nucleotide alterations. The latter, also known as point mutations, may frequently intervene in carcinogenesis, since a variety of mutagenic chemicals induce such mutations. In addition, such mutations may occur spontaneously as a result of mistakes in DNA replication. As used herein the term “mutant or mutated” as applied to a target neoplastic nucleotide sequence shall be understood to encompass a mutation, a restriction fragment length polymorphism, a nucleic acid deletion, or a nucleic acid substitution. A point mutation constitutes a single base change in a DNA strand, for example a G residue altered to a T. Such a mutation may alter the identity of the codon in which it lies thereby creating a missense mutation or nonsense mutation. Transition mutations involve the substitution of one purine in the DNA by another purine or one pyrimidine by another pyrimidine, that is A by G or vice versa, or T by C and vice versa. Transversions involve the replacement of a purine by a pyrimidine and vice versa.

A missense mutation is a point mutation in which a codon is changed into one encoding amino acid other than that normally found at a particular position. A nonsense mutation is any mutation that converts a codon specifying an amino acid into one coding for termination of translation. Such nonsense changes are usually accompanied by the loss of function of the gene product.

A splicing mutation is any mutation affecting gene expression by affecting correct RNA splicing. Splicing mutations may be due to mutations at intron-exon boundaries which alter splice sites. A polyadenylation site mutant is a mutation of the consensus sequence required for addition of poly (A) to the 3′ end of mature mRNA and which results in premature mRNA degradation.

An insertion is any mutation caused by the insertion of a nucleotide or stretch of nucleotides into a gene. For example, naturally occurring insertion mutations can be the result of the transposition of transposable genetic elements.

Regardless of the type of change, a change in the amino acid sequence is potentially detectable by antibodies, such as monoclonal antibodies developed against a particular peptide sequence.

Mutations that occur in somatic cells are not transmitted to the sexually produced offspring. However, such somatic mutations may be transferred to descendant daughter cells and mutations in some genes have been implicated in cancer. It is now clear that mutations may lead to the induction of cancer when they occur in one or more of a battery of normal genes referred to as the proto-oncogenes. Proto-oncogenes may be modified by a variety of mutational changes to produce the cancer-causing oncogenes. Proto-oncogenes play an essential part in the control of cell growth and differentiation and disruption of their normal activity by mutational events may lead to the aberrant growth characteristics observed in cancer cells.

The term “cancer,” encompasses any carcinoma in a tissue of a subject. Such carcinomas would include, for example, carcinoma of the mouth, esophagus, throat, larynx, thyroid gland, tongue, lips, salivary glands, nose, paranasal sinuses, nasopharynx, superior nasal valut and sinus tumors, esthesioneuroblastoma, squamous call cancer, malignant melanoma, sinonasal undifferentiated carcinoma (SNUC), or blood neoplasia. Also included are carcinomas of the regional lymph nodes, including cervical lymph nodes, prelaryngeal lymph nodes, pulmonary juxtaesophageal lymph nodes, and submandibular lymph nodes. Other carcinomas include carcinomas of the breast tissue or ducts. By subject is meant any mammal such as bovine, canine, feline, porcine and humans.

Treatment of cell proliferative disorders such as neoplasms and cancer are becoming more common with the development of molecular biology and the understanding of cell cycle regulation. However, it is important to be able to diagnose a cancerous condition as early in its development as possible. It would be beneficial to be able to diagnose a cell proliferative disorder when the cells having the disorder are not so numerous so as they become more difficult to treat and have a greater opportunity to metastasize.

The invention allows for diagnosis of cell proliferative disorders in biological samples containing a small percentage of cells having a disorder compared to the total number of cell in the sample (i.e., rare cells). The invention provides a method whereby cells in a sample eliciting markers of a cell proliferative disorder can be efficiently concentrated from total cell content of the sample. Accordingly, by identifying and concentrating these “rare” cells the invention provides the ability to more accurately diagnose a cell proliferative disorder in a subject from a small sample or in samples where cancerous cells are rare.

The invention provides methods, compositions, and kits that use antibodies (as described more fully below), which recognize makers on cells indicative of a cell proliferative disorder. These antibodies are capable of binding to markers on a cell having a cell proliferative disorder. The antibodies themselves can be bound to magnetic beads that are then used to separate the antibody-bound cells to concentrate them from the sample (e.g., by creating a “sub-sample”).

The invention further combines the enrichment techniques herein with an automated system for detecting rare events in a biological sample. The automated system utilizes an automated optical system and processing algorithms that are capable of identifying a single rare event (e.g., a rare cell type) in a sample comprising many other events (e.g., normal cells).

Kohler and Milstein are generally credited with having devised the techniques that successfully resulted in the formation of the first monoclonal antibody-producing hybridomas (G. Kohler and C. Milstein, Nature, 256:495-497 (1975); Eur. J. Immunol., 6:511-519 (1976)). By fusing antibody-forming cells (spleen lymphocytes) with myeloma cells (malignant cells of bone marrow primary tumors) they created a hybrid cell line, arising from a single fused cell hybrid (called a hybridoma or clone) which had inherited certain characteristics of both the lymphocytes and myeloma cell lines. Like the lymphocytes (taken from animals primed with sheep red blood cells as antigen), the hybridomas secreted a single type of immunoglobulin specific to the antigen; moreover, like the myeloma cells, the hybrid cells had the potential for indefinite cell division. The combination of these two features offered distinct advantages over conventional antisera. Whereas, antisera derived from vaccinated animals are variable mixture of polyclonal antibodies which never can be reproduced identically, monoclonal antibodies are highly specific immunoglobulins of single type. The single type of immunoglobulin secreted by a hybridoma is specific to one and only one antigenic determinant, or epitope, on the antigen, a complex molecule having a multiplicity of antigenic determinants. For instance, if the antigen is a protein, an antigenic determinant may be one of the many peptide sequences, generally 6-7 or more amino acids in length (M. Z. Atassi, Molec. Cell. Biochem., 32:21-43 (1980)), within the entire protein molecule. Hence, monoclonal antibodies raised against a single antigen may be distinct from each other, depending on the determinant that induced their formation; but for any given clone, all of the antibodies it produces are identical. Furthermore, the hybridoma cell line can be reproduced indefinitely, is easily propagated in vitro or in vivo, and yields monoclonal antibodies in extremely high concentration.

Monoclonal antibodies are presently being applied by investigators to the diagnosis and treatment of cancer (for a general discussion of the topic, see Hybridomas in Cancer Diagnosis and Treatment, Mitchell, M. S. and Oettgen, H. F., (eds.), Progress in Cancer Research and Therapy, Vol. 21, Raven Press, New York (1982)). Monoclonal antibodies have been raised against tumor cells (U.S. Pat. No. 4,196,265), carcinoembryonic antigen (U.S. Pat. No. 4,349,528), and thymocytes, prothymocytes, monocytes, and suppressor T cells (U.S. Pat. Nos. 4,364,933; 4,364,935; 4,364,934; 4,364,936; 4,364,937; and 4,364,932). Recent reports have demonstrated the production of monoclonal antibodies with various degrees of specificity to several human malignancies, including mammary tumor cells (Colcher, D. et al., Proc. Natl. Acad. Sci. U.S.A., 78:3199-3203 (1981)), lung cancers (Cuttitta, F. et al., Proc. Natl. Acad. Sci: U.S.A., 78:495-4595 (1981)), malignant melanoma (Dippold, W. G. et al., Proc. Natl. Acad. Sci. U.S.A., 77:6114-6118 (1980)), colorectal carcinoma (Herlyn, M. et al., Proc. Natl. Acad. Sci. U.S.A., 76:1438-1442 (1979)), lymphoma (Nadler, L. M. et al., J. Immunol., 125:570-577 (1980)), and neuroectodermal tumors (Wikstrand, C. J. and Bigner, D. C., Cancer Res., 43:267-275 (1982)).

Several investigators have reported on the production of monoclonal antibodies against epitopes of various normal and malignant mammary cell components. (Arklie, J. et al., Int. J. Cancer, 28:23-29 (1981); Ciocca, D. R. et al., Cancer Res., 42;4256-4258 (1982); Colcher, D. et al., Proc. Natl. Acad. Sci. U.S.A., 78:3199-3203 (1981); Foster, C. S., et al., Virchows Arch. Pathol. Anat., 394:279-293 (1982); Greene, G. L. et al., Proc. Natl. Acad. Sci. U.S.A., 77:5115-5119 (1980); McGee, JO'D. et al., Lancet, 2:7-11 (1982); Nuti, M. et al., Int. J. Cancer, 291:539-545 (1982); and Taylor-Papadimitriou, J. et al., Int. J. Cancer, 28:17-21(1981)). Many of the antigens recognized above are differentiation-related; therefore, these antibodies are most suited to histologically assess the differentiated status or grade of tumor specimens. For example, monoclonal antibodies directed against several antigens of human milk-fat-globule membranes have been produced. These antibodies have proven useful in studying the derivation of cell cultures, in evaluating the phenotypic expression of antigens in neoplastic transformation, have served as differentiation markers in breast cancer, and as immunodiagnostic reagents in the quantitation of antigens in the sera of breast cancer patients (Arklie, J. et al., Int. J. Cancer, 28:23-29(1981); Ceriani, R. L. et al., Proc. Natl. Acad. Sci. U.S.A., 74:582-586(1977); Ceriani, R. L. et al., Proc. Natl. Acad. Sci., 79:5420-5424 (1982); Foster, C. S. et al., Virchows Arch. Pathol. Anat., 394:279-293(1982); and Taylor-Papadimitriou, J. et al., Int. J. Cancer, 28:1721(1981)).

Arklie et al. have described monoclonal antibodies directed against human milk-fat-globule membranes. These antibodies showed a stronger staining reaction with well-differentiated (grade I) ductal carcinomas than undifferentiated (grade III) tumors. Nuti et al. have produced monoclonal antibodies against human metastatic breast carcinoma cells, which have been used to indicate tumor antigen heterogeneity. Foster et al. have also reported the production of monoclonal antibodies, which were used to show significant heterogeneity of antigen expression within breast tumors. Other reported monoclonal antibodies directed against carcinoma-associated antigens are identifiable by those of skill in the art.

The invention provides methods and compositions for enriching cancer cells in a sample. One method employs positive selection and utilizes the binding affinity of antibodies directed to cell surface markers indicative of a cancer phenotype to purify these cells from non-cancer cells. Such techniques may employ column fractionation or affinity purification protocols.

An alternative carcinoma cell enrichment method, named negative selection, is based on the depletion of non-tumor cells present in a sample. This method utilizes antibodies directed to one or several cell surface markers expressed by noncarcinoma cells, such as CD45 expressed by white blood cells. The negative selection method offers the advantage of not relying on the presence of a carcinoma cell surface marker. These markers can have a wide range of expression due to the diversity of tumor cell prototypes.

Using the techniques and compositions described generally above, the Applicant has developed a method of enriching the number of neoplastic cells in a sample, using both positive and negative selection sequentially, to maximize the sensitivity of the carcinoma cell detection. Alternatively, each method (positive and negative selections) can be used alone.

In one aspect of the invention, the enriched sample is then placed on a slide or other substrate that is optically transmissive (e.g., such as glass). In another aspect of the invention, the enriched sample is placed on a substrate that may or may not be optically transmissive to light.

The method couples composite images in an automated manner for processing and analysis. A slide on which is mounted an enriched sample stained to identify a structure, cell, or event of interest is supported on a motorized stage. An image of the biological sample is generated, digitized, and stored . As the viewing field of the objective lens is smaller than the entire sample, a histological reconstruction is made. These stored images of the entire sample can then be analyzed using the algorithms described herein to identify a structure, cell or event of interest (e.g., a rare event).

In one embodiment, the invention provides a method for automated image analysis of an enriched sample by providing a sample enriched as described above to be analyzed, automatically scanning the sample at a plurality of coordinates, automatically obtaining an image at each of the coordinates, reconstructing an image of the sample from each individual image to create a reconstructed image and processing the reconstructed image to identify a rare event.

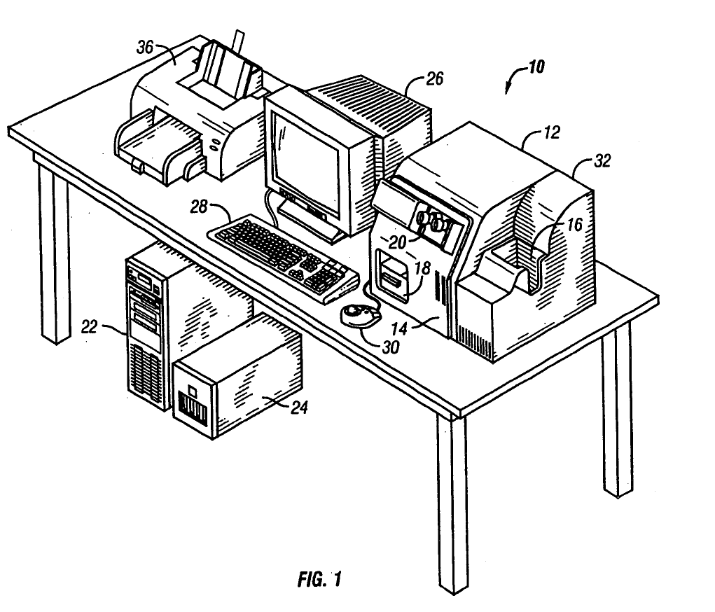

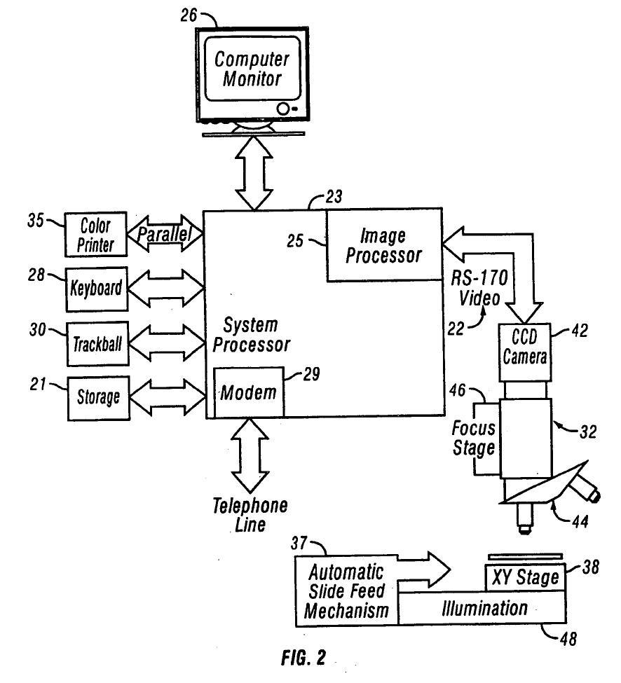

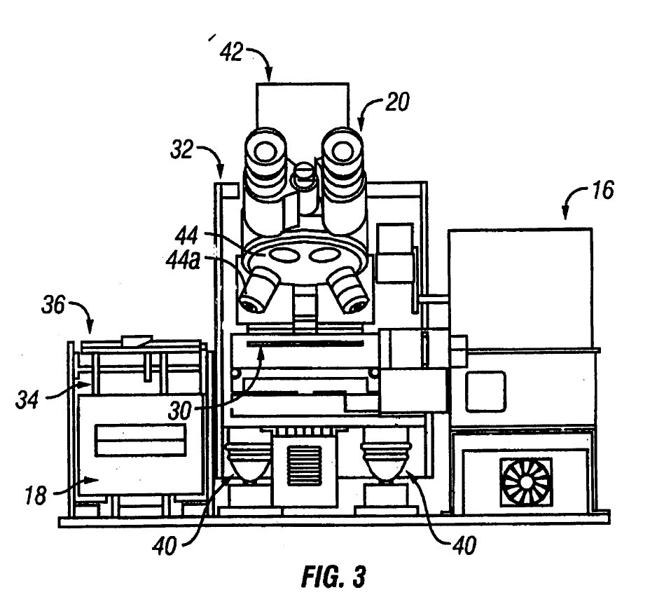

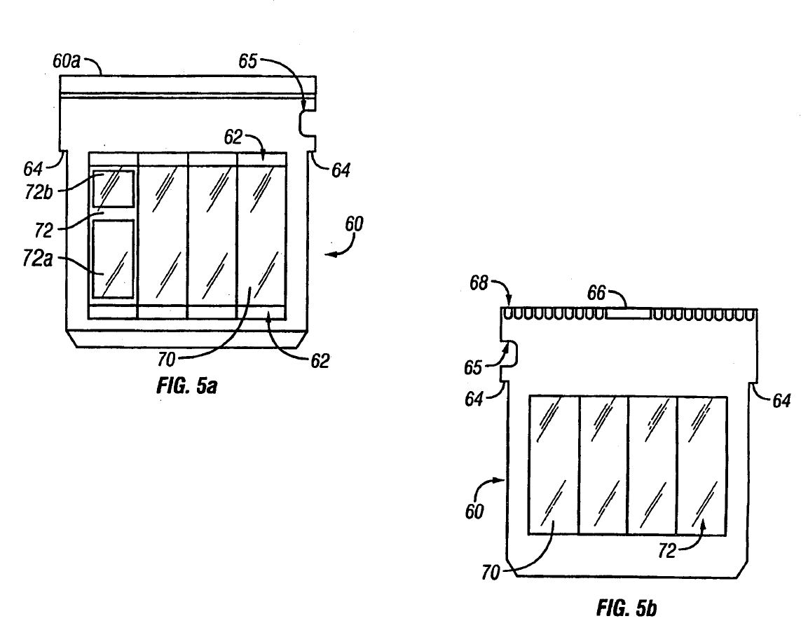

An automated microscope for analyzing the enriched sample is shown in FIGS. 1 and 3 and in block diagram in FIG. 2. A motorized stage 38 may be used to support a slide 70 (FIG. 5). On the slide is mounted an enriched sample that is typically stained to identify a structure of interest for analysis (e.g., a particular marker of a rare event). An enriched sample comprises a cellular or an acellular sample of biological origin that has been enriched through positive and/or negative selection for a particular molecule or cell types. The enriched sample is mounted on a substrates such as a microscope slide. The enriched sample can be, for example, derived from a biological fluid sample, for example, a blood fraction cytospun on a microscope slide or an enriched cell suspension applied directly on a slide.

At least one lens, such as an objective lens, 44a is located above the stage and a light source 48 is located beneath the stage (e.g., in transmitted light analysis). Light from the source illuminates the stage and slide so an image of the sample is generated by the objective lens. This image is stored in memory. Typically the image is a digitized or digital image. As the viewing field of the lens is smaller than the entire sample, the stage is moved in one planar direction by a distance that corresponds to the length of the field of view in that direction. The image generated at that position may then be captured and stored. The acquired image may be flipped along its centerline due to the optical flipping of the original image. Movement of the stage and capture of the resulting image continues in the same direction until the end of the sample area of the slide is reached. At that time, the stage is moved in the other planar direction by a distance that corresponds to the length of the field of view in that direction and another image is generated and stored. The slide is traversed or scanned in this manner until the entire sample area of the slide has been viewed through the objective lens. These stored images may then be placed together in the order in which they were collected to generate a composite or reconstructed image of the sample. This composite image may then be analyzed to detect a structure (e.g., a rare event) that extends across more than one image field or more than one slide for further analysis. Such analysis may result in the identification of a candidate object or area of interest (e.g., a rare event) in both the field of view as well as objects that overlap two or more fields of view. In such instances, the system will automatically determine the coordinates for these candidate objects and may obtain additional images at various magnifications.

The methods of the invention are capable of identifying structures in a sample that cannot be captured in a single field of view image. The methods of the invention use an analysis technique to identify field of view images that appear to contain part of a tissue structure to be analyzed. Field of view images so identified that are adjacent to one another are then identified as containing the tissue structure that the stain, antibody, or probe was intended to identify. This portion of the composite image may then be viewed under a higher magnification power for additional detail.

Nuclear Stains, Intercalating Dyes and Counterstains are used in the imaging process of the methods of the invention. The term “nuclear stain” refers to a cytochemical stain that preferentially stains the nuclei of eukaryotic cells. Many nuclear stains are intercalating dyes. The term “intercalating dye” refers to a chemical compound that can insert itself in between adjacent nucleotides of a nucleic acid to provide a detectable color.

Many nuclear stains are known in the art, with one of the most commonly used being hematoxylin. Hematoxylin is often used in combination with various metallic salts (mordants). Hematoxylin stains are used for different staining purposes, and have a variety of colors, depending on the metal used. Aluminum lakes are purple to blue, depending on pH. Iron lakes are blueblack. Chromium lakes are blue-black. Copper lakes are bluegreen to purple. Nickel lakes are various shades of violet. Tin lakes are red. Lead lakes are dark brown. Osmium lakes are greenish brown. Other nuclear stains include Giemsa stain, methyl green (which binds to AT-rich DNA regions), and Nuclear Fast-Red.

Fluorescent stains include Hoechst 33342; Hoechst 33258 (Calbiochem), a bisbenzimide DNA intercalator that excites in the near UV (350 nm) and emits in the blue region (450 nm); thiazole orange, a fluorogenic stain for DNA that excites in the blue region (515 nm) and emits in the green region (530 nm) of the visible spectrum; DAPI; ethidium bromide; propidium iodide; TOTO; YOYO-1; and SYTOX Blue or Green stains are also encompassed by the current invention. Several dyes either bind GC-rich or AT-rich chromosomal regions preferentially or show differences in fluorescence intensity upon binding those regions, yielding fluorescent banding patterns. For example, 7-Aminoactinomycin D binds selectively to GC-rich DNA regions and. 9-Amino-6-chloro-2-methoxyacridine fluoresces with greatest intensity in AT-rich DNA regions. Acridine homodimer fluoresces preferentially when bound to AT-rich DNA regions.

The term “counterstain,” when used in combination with nuclear stains, refers to cytochemical stains that bind to a region of a eukaryotic cell other than the nucleus. Many counterstains are known in the art. One of the most common is eosin, which stains eukaryotic cell cytoplasm to varying shades of pink. Other counterstains are specific for a particular organelle or a protein in a cell. For example, the Kleihauer-Betke cytochemical stain is specific for hemoglobin F, a hemoglobin type preferentially expressed in fetal cells and therefore can be defined as a specific marker of fetal red blood cells.

The term “coordinate” or “address” is used to mean a particular location on a slide or sample. The coordinate or address can be identified by any number of means including, for example, X-Y coordinates, r-θ coordinates, and others recognized by those skilled in the art.

In one embodiment, an automated cellular imaging method is used to identify fetal nucleated red blood cells in a maternal blood sample. Fetal cells are first enriched through positive or negative selection methods as described herein. The enriched sample is the stained for the rare cell event (e.g., fetal nucleated red blood cells). For example, the enriched sample is stained with a Kleihauer-Betke cytochemical stain. Kleihauer-Betke cytochemically stained cells (e.g., hemoglobin F. Fetal cells) are identified by the automated cellular imaging system as objects on the basis of their bright red color (indicative of Hemoglobin F) as compared to maternal red blood cells. To assure that appropriate objects are identified, size and shape morphological “filters” are used to exclude very small and very large objects.

Cells are counterstained with an additional cytochemical stain for nucleic acids, resulting in a blue color for nucleated red blood cells (e.g., fetal red blood cells). An automated image analysis system identifies blue objects of the appropriate size and shape for an erythrocyte nucleus among the bright red objects, allowing the imaging system to identify and enumerate nucleated fetal red cells. Such cells can be enumerated, allowing for a screen for Down's syndrome in the fetus, wherein the frequency of such cells is typically higher in Down's syndrome pregnancies compared with normal pregnancies.

The results of the hematoxylin/eosin (H/E) staining provide cells with nuclei stained blue-black, cytoplasm stained varying shades of pink; muscle fibers stained deep pinky red; fibrin stained deep pink; and red blood cells stained orangered.

For example, H/E slides are prepared with a standard H/E protocol. Standard solutions include the following: (1) Gills hematoxylin (hematoxylin 6.0 g; aluminum sulphate 4.2 g; citric acid 1.4 g; sodium iodate 0.6 g; ethylene glycol 269 ml; distilled water 680 ml); (2) eosin (eosin yellowish 1.0 g; distilled water 100 ml); (3) lithium carbonate 1% (lithium carbonate 1 g; distilled water 100 g); (4) acid alcohol 1% 70% (alcohol 99 ml conc.; hydrochloric acid 1 ml); and (5) Scott's tap water. In a beaker containing 1 L distilled water, add 20 g sodium bicarbonate and 3.5 g magnesium sulphate. Add a magnetic stirrer and mix thoroughly to dissolve the salts. Using a filter funnel, pour the solution into a labeled bottle.

The staining procedure is as follows: (1) Bring the tissue or cell sections to water; (2) place sections in hematoxylin for 5 minutes (min); (3) wash in tap water; (4) ‘blue’ the sections in lithium carbonate or Scott's tap water; (5) wash in tap water; (6) place sections in 1% acid alcohol for a few seconds; (7) wash in tap water; (8)place sections in eosin for 5 min; (9) wash in tap water; and (10) dehydrate with graded alcohol solution. Mount sections.

A specific marker is a molecule or a group of molecules, which is/are present in only a subset of the components of a biological sample and therefore identifying specifically the components having the marker. Specific markers are frequently defined as antigens recognized by specific antibodies (monoclonals or polyclonals) and can be detected by immunohistochemistry.

Another group of specific markers is defined by the capacity of these markers to hybridize, specifically, a nucleic acid probe. These markers can usually be detected by in situ hybridization.

A third group of specific markers can be defined by their enzymatic activity and can be detected by histochemistry.

A fourth group of specific markers can be stained directly, histochemically, using a specific dye.

A fifth group of specific markers can be defined as being receptors binding specifically to one or several ligands. A specific ligand is itself used for the detection of the receptor-ligand complex, using a detection method involving either histochemistry, or immunohistochemistry or in situ hybridization.

Immunohistochemical techniques as used herein encompasses the use of reagents detecting cell specific markers, such reagents include, for example, antibodies and nucleic acid probes. Antibodies, including monoclonal antibodies, polyclonal antibodies and fragments thereof, are often used to identify proteins or polypeptides of interest in a sample. A number of techniques are utilized to label objects of interest according to immunohistochemical techniques. Such techniques are discussed in Current Protocols in Molecular Biology, Unit 14 et seq., eds. Ausubel, et al., John Wiley & Sons, 1995, the disclosure of which is incorporated herein by reference. For example, the following procedure is an example of immunohistochemical staining using an antibody recognizing, specifically, the HER2 protein. HER2 overexpression has been described as a specific marker in a high percentage of breast cancer carcinomas.

As described above, antibodies are also used in the methods of the invention to enrich a sample by positive and/or negative selection techniques. In the immunohistochemical staining techniques described herein, the same or different antibodies can be used compared to the enrichment technique. There is an advantage to utilizing antibodies that specifically bind to a different epitope of a marker protein. For example, the use of different antibodies that recognize the same marker but bind to different epitopes on the marker are useful in avoiding false positives and/or cross recognition between various proteins. Thus, in one aspect of the invention a first antibody is used to positively select a particular cell type during the enrichment of the sample through the interaction of the first antibody with a first epitope on a cell maker. A second antibody is then used to immunohistochemically stain the enriched sample. The second antibody binds to the same marker, however it binds to an epitope that is different than the first antibody.

Immunohistochemical localization of cellular molecules uses the ability of antibodies to bind specific antigens, for example proteins of interest such as onco-proteins and enzymes, with high affinity. These antibodies can be used to localize antigens to subcellular compartments or individual cells within a sample.

In situ hybridization techniques include the use of specifically labeled nucleic acid probes, which bind to cellular RNA or DNA in individual cells or tissue section. Suitable nucleic acid probes may be prepared using standard molecular biology techniques including subcloning, plasmid preparation, and radiolabeling or non-radioactive labeling of the nucleic acid probe.

Immunofluorescent labeling of a sample often uses a sandwich assay or a primary antibody and secondary antibody-fluorochrome conjugate. An enriched sample suspected of containing rare event cells are first washed in phosphate buffered saline and then exposed to a primary antibody which will bind to a marker associated with the rare-event. Subsequently the cells of the enriched sample are washed and exposed to the secondary antibody which binds to the first or primary antibody. The cells of the enriched sample are washed and cytospun or otherwise place on a slide. Numerous other techniques well known in the art of immunohistochemical staining and in situ hybridization are easily adaptable for use in immunohistochemical reconstruction as disclosed herein. Thus, a combination of techniques using both chemical staining and/or immunohistochemical and/or in situ hybridization may be used in the present methods.

Histological reconstruction is a process whereby an image of a whole sample is constructed from analyzed pieces of the sample, particularly when the sample has been mounted on a slide. This image is created by piecing together more than one field of view at any particular magnification.

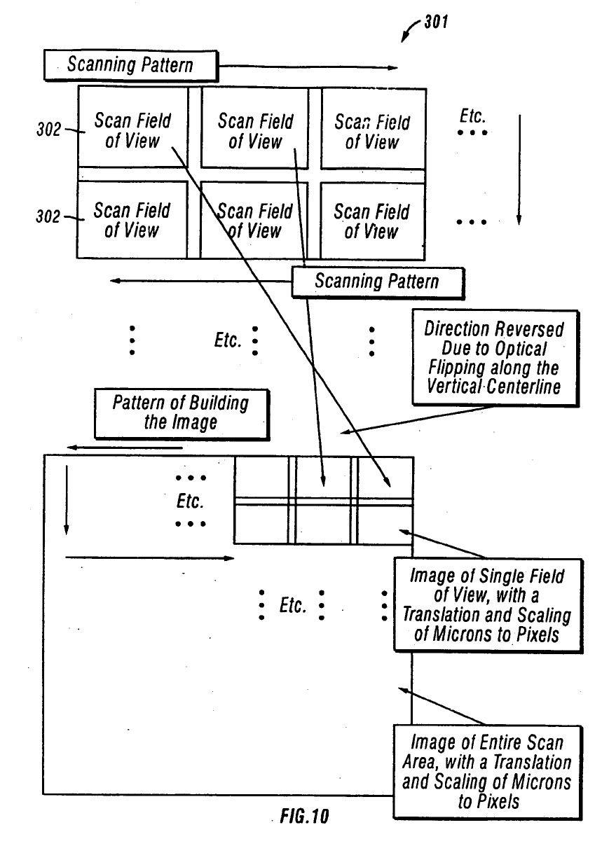

With reference to FIG. 10, an image 302, representing an objective's field of view is acquired at a first particular coordinate on the slide sample 301. The slide is automatically repositioned on the X-Y stage to obtain a new or second field of view corresponding to a second particular coordinate 303. This new field of view is preferably immediately adjacent to the first field of view, however, so long as the coordinates, thus the address/identity, of each field of view are retained in the imaging system, histological reconstruction may be performed. This process is repeated until images for the whole of the sample have been acquired.

Based upon each image's X and Y coordinate, the sample is digitally reconstructed. As part of the reconstruction, the image may be flipped to correct for the optical flipping of the original image.

The process of forming a histological reconstructed image involves having the apparatus scan a microscope slide of interest, and form the image that constitutes a reconstruction of the images taken during the scan. The image that is formed can be a full-color reconstruction of the entire scan area, or a fraction of the whole scan area, for example, reconstruction of the entire scan area that identifies objects or areas of interests. The reconstructed digital image can then be used for further processing or analysis to identify previously undetected objects or areas of interest (e.g., rare events). For example, objects or areas of interest overlapping one or more fields of view or slides may thus be identified in the reconstructed digital image.

With reference to FIGS. 1 and 2, the apparatus 10, also referred to as the system, comprises a microscope 32 with a motorized X,Y and Z stage 38, a camera 42, a computer 22 adapted to receive and process video images, and a set of software programs to control the apparatus and to execute the method. A measurement of the optical properties of the sample features is used to form an image of the scannable area of the slide, to find sub-regions of interest, and to analyze the properties of these regions. The image processing method that evaluates the sample to find regions of interest uses a measure of the hue, saturation and/or intensity and luminosity of a 24-bit color image to produce a white on black target image of interest. This image is processed by separately converting the full color image (red, green, blue -RGB) to components of hue, saturation or intensity and luminosity, thresholding the components, and performing a logical “AND” between the two images, then thresholding the resulting image such that any pixel value above zero becomes 255. The processing and image acquisition will be further understood with reference to the apparatus described below.

With reference to FIG. 1, a slide prepared with an enriched sample and a reagent (e.g., an agent that specifically stains or interacts with a marker) is placed in a slide carrier 60 (FIG. 5) can hold from 1-10 slides, but typically holds four slides. The slide carriers are loaded into an input hopper 16 of the automated system 10. The operator then enters data identifying the instrument protocol which contains information on the size, shape and location of a scan area on each slide, or, preferably, the system automatically locates a scan area for each slide during slide processing. The operator then activates the system 10 for slide processing. At system activation, a slide carrier 60 is positioned on an X-Y stage 38 of an optical system, such as microscope subsystem 32. Any bar codes used to identify slides are read and stored for each slide in the carrier. The entire slide is rapidly scanned at a low magnification, typically 10×. At each location of the scan, a low magnification image is acquired and processed to detect candidate objects or areas of interest. Typically, color, size and shape are used to identify objects or areas of interest. The location of each candidate object or area of interest may be stored by reference to its coordinates or address. Each field of view may also be stored as part of a larger composite image.

At the completion of the low level scan for each slide in the carrier on the stage, the optical system may be adjusted to a higher magnification such as 40× or 60×, for additional sample processing and image acquisition, and the X-Y stage is positioned to the stored locations for the candidate objects or areas of interest on each slide in the carrier. A higher magnification image is acquired for each candidate object or area of interest and a series of image processing steps are performed to confirm the analysis, which was performed at low magnification. A higher magnification image is stored for each continued object or area of interest. These images are then available for retrieval by a pathologist or cytotechnologist to review for final diagnostic evaluation. Having stored the location of each object or area of interest, a mosaic comprising the candidate objects or areas of interest for a slide may be generated and stored. The pathologist or cytotechnologist may view the mosaic or may also directly view the slide at the location of an object or area of interest in the mosaic for further evaluation. The mosaic may be stored on magnetic or optical media for future reference or may be transmitted to a remote site for review or storage. The entire process involved in examining a single slide takes on the order of 4-100 min depending on scan area size and the number of detected candidate objects of interest.

The processing of images acquired in the automated scanning includes the steps of transforming the image to a different color space, such as hue, saturation and intensity. The pixels of the filtered image are dynamically thresholded to suppress background material; performing a morphological function to remove artifacts from the thresholded image; analyzing the thresholded image to determine the presence of one or more regions of connected pixels having the same color; and categorizing every region having a size greater than a minimum size as a candidate object or area of interest.

According to another aspect, the scan area is automatically determined by scanning the slide; acquiring an image at each slide position; analyzing texture or color information for each image to detect the edges of the sample and storing the locations corresponding to the detected edges to define the scan area.

According to yet another aspect, automated focusing of the optical system is achieved by initially determining a focal surface from an array of points or locations in the scan area. The derived focal surface enables subsequent rapid automatic focusing in the low power scanning operation. In one embodiment, the focal plane is determined by determining proper focal positions across an array of locations and performing a least squares fit of the array of focal positions to yield a focal plane across the array. Typically, a focal position at each location is determined by incrementing the position of a Z stage for a fixed number of coarse and fine iterations. At each iteration, an image is acquired and a pixel variance, morphological gradient or other optical parameter about a pixel mean for the acquired image is calculated to form a set of evaluation data. The peak value of the least squares fit curve is selected as an estimate of the best focal position.

In another aspect, a focal position method for a higher magnification locates a region of interest centered about a candidate object of interest/rare event within a slide which was located during an analysis of the low magnification images. The region of interest is preferably n columns wide, where n is a power of 2. The pixels of this region are then processed using a Fast Fourier Transform to generate a spectra of component frequencies and corresponding complex magnitude for each frequency component. The complex magnitude of the frequency components which range from 25% to 75% of the maximum frequency component are squared and summed to obtain the total power for the region of interest. This process is repeated for other Z positions and the Z position corresponding to the maximum total power for the region of interest is selected as the best focal position. This focal method can be used with many stains and types of cellular samples.

The handling of the slide comprising the enriched sample may be processed automatically. A slide is mounted onto a slide carrier 60 (FIG. 5) with a number of other slides side-by-side. The slide carrier 60 is positioned in an input feeder 16 with other slide carriers to facilitate automatic analysis of a batch of slides. The slide carrier is loaded onto the X-Y stage 38 of the optical system 32 for the analysis of the slides thereon. Subsequently, the first slide carrier is unloaded into an output feeder 18 after automatic image analysis and the next carrier is automatically loaded.

Referring to the Figures, an apparatus for automated cell image analysis of biological samples is generally indicated by reference numeral 10 as shown in perspective view in FIG. 1 and in block diagram form in FIG. 2. The apparatus 10 comprises a microscope subsystem 32 housed in a housing 12. The housing 12 includes a slide carrier input hopper 16 and a slide carrier output hopper 18. A door 14 in the housing 12 secures the microscope subsystem from the external environment. A computer subsystem comprises a computer 22 having two system processors 23, an image processor 25 and a communications modem 29. The computer subsystem further includes a computer monitor 26 and an image monitor 27 and other external peripherals including storage device 21, pointing device 30, keyboard 28 and color printer 35. An external power supply 24 is also shown for powering the system. Viewing oculars 20 (optional) of the microscope subsystem project from the housing 12 for operator viewing. The apparatus 10 further includes a CCD camera 42 for acquiring images through the microscope subsystem 32. The computer directly controls a number of microscope subsystem functions described further in detail.

An automatic slide feed mechanism 37 in conjunction with X-Y stage 38 provide automatic slide handling in the apparatus 10. An illumination light source 48 projects light onto the X-Y stage 38 which is subsequently imaged through the microscope subsystem 32 and acquired through the CCD camera 42 for processing by the image processor 25. A Z stage or focus stage 46 under control of the microscope controller 31 provides displacement of the microscope subsystem in the Z plane for focusing. The microscope subsystem 32 further includes a motorized objective turret 44 for selection of objectives.

The apparatus 10 is for the unattended automatic scanning of prepared microscope slides for the detection and counting of candidate objects (rare events) or areas of interest, such as stained cells. In one embodiment, rare event detection in which there may be only one candidate object of interest per several hundred thousand normal cells, e.g., one to five candidate objects of interest per 2 square centimeter area of the slide. The apparatus 10 automatically locates and counts candidate objects or areas of interest and estimates normal cells present in a cellular sample on the basis, for example, of color, size and shape characteristics. A sample may be prepared with a reagent to obtain a colored insoluble precipitate. The apparatus, in one embodiment, is used to detect this precipitate as a candidate object or area of interest.

During operation of the apparatus 10, a pathologist or laboratory technician mounts prepared slides onto slide carriers. A slide carrier 60 is illustrated in FIG. 5 and is described further below. Each slide carrier holds a plurality of slides (a 4 slide carrier is shown in FIG. 5). Up to 25 slide carriers are then loaded into input hopper 16. The operator can specify the size, shape and location of the area to be scanned or alternatively, the system can automatically locate this area. The operator then commands the system to begin automated scanning of the slides through a graphical user interface. Unattended scanning begins with the automatic loading of the first carrier and slide onto the motorized X-Y stage 38. A bar code label affixed to the slide is read by a bar code reader 33 during this loading operation. Each slide is then scanned at a user selected low microscope magnification, for example, lox, to build a histological reconstruction or identify candidate objects based on their color, size and shape characteristics. The X-Y locations of candidate objects or areas of interest are stored until scanning is completed.

After the low magnification scanning is completed, the apparatus may automatically return to each candidate object or area of interest, if necessary, reimaging and refocusing at a higher magnification such as 40× and performs further analysis to confirm the biological candidate/object. The apparatus stores an image of the object or area of interest for later review by a pathologist. All results and images can be stored to a storage device 21 such as a removable hard drive or optical disc or DAT tape or transmitted to a remote site for review or storage. The stored images for each slide can be viewed in a mosaic of images for further review. In addition, the pathologist or operator can also directly view a detected object or area of interest through the microscope using the oculars 20 (optional)or on image monitor 27.

One or more system processors may be present. The system processor(s) 102 further controls an illumination controller 106 for control of substage illumination 48. The light output from, for example, a halogen light bulb, which supplies illumination for the system, can vary over time due to bulb aging, changes in optical alignment, and other factors. In addition, slides which have been “over-stained” can reduce the camera exposure to an unacceptable level. To compensate for these effects, the illumination controller 106 is included. This controller is used in conjunction with light control software to compensate for the variations in light level. The light control software samples the output from the camera at intervals (such as between loading of slide carriers), and commands the controller to adjust the light level to the desired levels. In this way, light control is automatic and transparent to the user and adds no additional time to system operation.

The system processor(s) 23 is comprised of the latest version of, for example, the Intel processors (e.g., an Intel Pentium IV). Where more than one processor is used the system may comprise dual parallel Intel Pentium IV 2 GHZ devices. The image processor 25 is preferably a Matrox Genesis board. The computer will typically operate under Windows NT, although other operating systems may be used. It will be recognized that any number of processors and operating systems can be used in the methods and in conjunction with the invention.

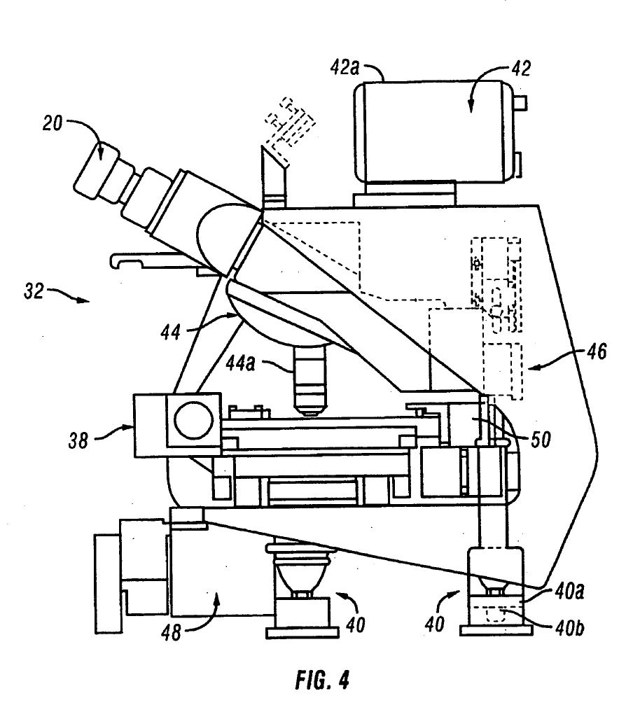

Referring now to FIG. 3 and 4, further detail of the apparatus 10 is shown. FIG. 3 shows a plan view of the apparatus 10 with the housing 12 removed. A portion of the automatic slide feed mechanism 37 is shown to the left of the microscope subsystem 32 and includes slide carrier unloading assembly 34 and unloading platform 36 which in conjunction with slide carrier unloading hopper 18 function to receive slide carriers which have been analyzed.

Vibration isolation mounts 40, shown in further detail in FIG. 4, are provided to isolate the microscope subsystem 32 from mechanical shock and vibration that can occur in a typical laboratory environment. In addition to external sources of vibration, the high-speed operation of the X-Y stage 38 can induce vibration into the microscope subsystem 32. Such sources of vibration can be isolated from the electro-optical subsystems to avoid any undesirable effects on image quality. The isolation mounts 40 comprise a spring 40a and piston 40b submerged in a high viscosity silicon gel which is enclosed in an elastomer membrane bonded to a casing to achieve damping factors on the order of 17% to 20%.

The automated slide handling subsystem operates on a single slide carrier at a time. A slide carrier 60 is shown in FIGS. 5a and 5b, which provide a top view and a bottom view, respectively. The slide carrier 60 can include a plurality of slides (e.g., 2-10) and is depicted as including up to four slides 70. The carrier 60 includes ears 64 for hanging the carrier in the output hopper 18. An undercut 66 and pitch rack 68 are formed at the top edge of the slide carrier 60 for mechanical handling of the slide carrier. A keyway cutout 65 is formed in one side of the carrier 60 to facilitate carrier alignment. A prepared slide 72 mounted on the slide carrier 60 includes a sample area 72a and a bar code label area 72b.

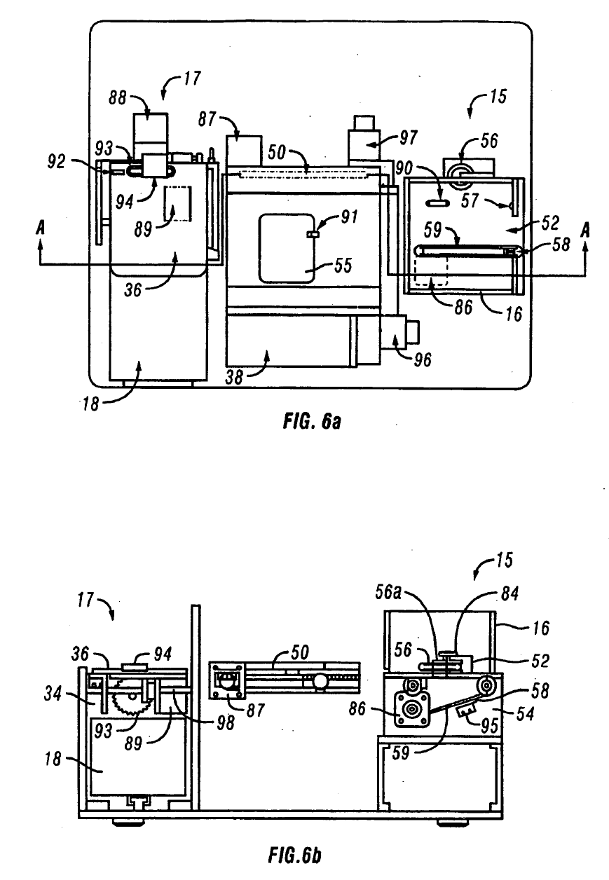

FIG. 6a provides a top view of the slide handling subsystem which comprises a slide input module 15, a slide output module 17 and X-Y stage drive belt 50. FIG. 6b provides a partial cross-sectional view taken along line A-A of FIG. 6a.

The slide input module 15 includes a slide carrier input hopper 16, loading platform 52 and slide carrier loading subassembly 54. The input hopper 16 receives a series of slide carriers 60 (FIG. 5a and 5b) in a stack on loading platform 52. A guide key 57 protrudes from a side of the input hopper 16 to which the keyway cutout 65 (FIG. 5a) of the carrier is fit to achieve proper alignment.

The input module 15 further includes a revolving indexing cam 56 and a switch 90 mounted in the loading platform 52, the operation of which is described further below. The carrier subassembly 54 comprises an infeed drive belt 59 driven by a motor 86. The infeed drive belt 59 includes a pusher tab 58 for pushing the slide carrier horizontally toward the X-Y stage 38 when the belt is driven. A homing switch 95 senses the pusher tab 58 during a revolution of the belt 59.

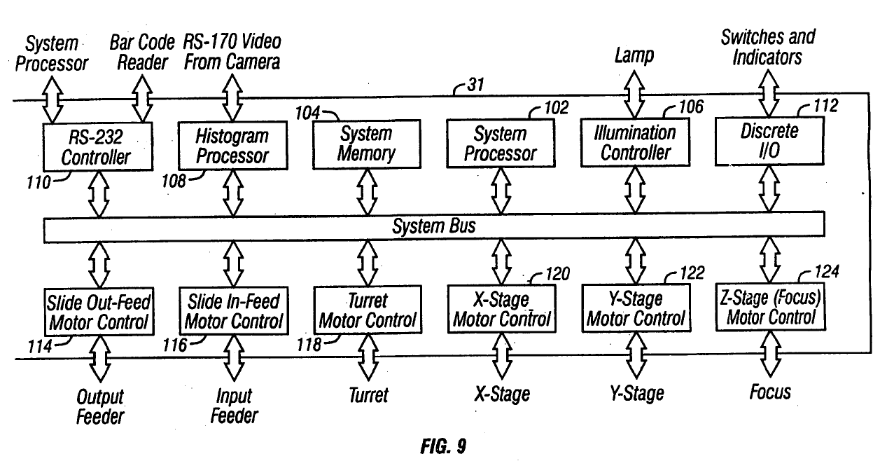

Referring specifically to FIG. 6a, the X-Y stage 38 is shown with x position and y position motors 96 and 97, respectively, which are controlled by the microscope controller 31 (FIG. 9). The X-Y stage 38 further includes an aperture 55 for allowing illumination to reach the slide carrier. A switch 91 is mounted adjacent the aperture 55 for sensing contact with the carrier and thereupon activating a motor 87 to drive stage drive belt 50 (FIG. 6b). The drive belt 50 is a double-sided timing belt having teeth for engaging pitch rack 68 of the carrier 60 (FIG. 5b).

The slide output module 17 includes slide carrier output hopper 18, unloading platform 6, and slide carrier unloading subassembly 34. The unloading subassembly 34 is a motor 89 for rotating the unloading platform 36 about shaft 98 during an unloading operation described further below. An outfeed gear 93 driven by motor 88 rotatably engages the pitch rack 68 of the carrier 60 (FIG. 5b) to transport the carrier to a rest position against switch 92. A spring loaded hold-down mechanism holds the carrier in place on the unloading platform 36.

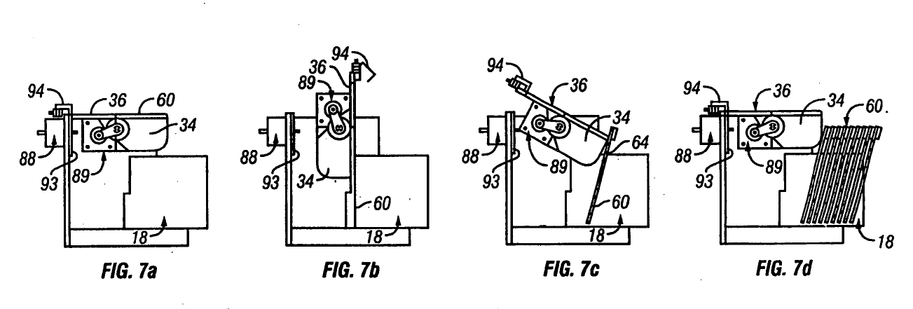

The slide handling operation is now described. Referring to FIG. 7, a series of slide carriers 60 are shown stacked in input hopper 16 with the top edges 60a aligned. As the slide handling operation begins, the indexing cam 56 driven by motor 85 advances one revolution to allow only one slide carrier to drop to the bottom of the hopper 16 and onto the loading platform 52.

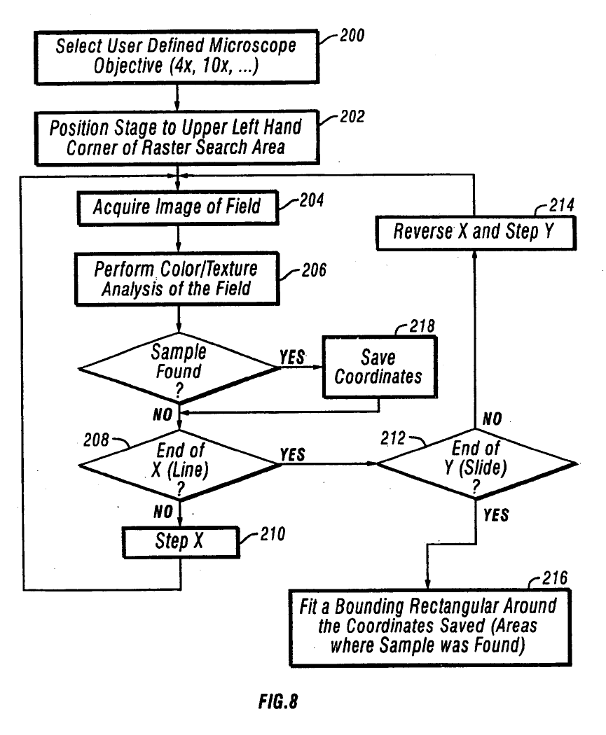

FIGS. 7a-7d show the cam action in more detail. The indexing cam 56 includes a hub 56a to which are mounted upper and lower leaves 56b and 56c respectively. The leaves 56b and 56c are semicircular projections oppositely positioned and spaced apart vertically. In a first position shown in FIG. 8a, the upper leaf 56b supports the bottom carrier at the undercut portion 66. At a position of the indexing cam 56 rotated 180°, shown in FIG. 7b, the upper leaf 56b no longer supports the carrier and instead the carrier has dropped slightly and is supported by the lower leaf 56c. FIG. 8c shows the position of the cam 56 rotated 2701 wherein the upper leaf 56b has rotated sufficiently to begin to engage the undercut 66 of the next slide carrier while the opposite facing lower leaf 56c still supports the bottom carrier. After a full rotation of 360° as shown in FIG. 7d, the lower leaf 56c has rotated opposite the carrier stack and no longer supports the bottom carrier which now rests on the loading platform 52. At the same position, the upper leaf 56b supports the next carrier for repeating the cycle.

Referring again to FIG. 6a and 6b, when the carrier drops to the loading platform 52, the contact closes switch 90 which activates motors 86 and 87. Motor 86 drives the infeed drive belt 59 until the pusher tab 58 makes contact with the carrier and pushes the carrier onto the X-Y stage drive belt 50. The stage drive belt 50 advances the carrier until contact is made with switch 91, the closing of which begins the slide scanning process described further herein. Upon completion of the scanning process, the X-Y stage 38 moves to an unload position and motors 8, and 88 are activated to transport the carrier to the unloading platform 36 using stage drive belt 50. Motor 88 drives outfeed gear 93 to engage the carrier pitch rack 68 of the carrier 60 (FIG. 5b) until switch 92 is contacted. Closing switch 92 activates motor 89 to rotate the unloading platform 36.

The unloading operation is shown in more detail in end views of the output module 17 (FIG. 7a-7d). In FIG. 7a, the unloading platform 36 is shown in a horizontal position supporting a slide carrier 60. The hold-down mechanism 94 secures the carrier 60 at one end. FIG. 7b shows the output module 17 after motor 89 has rotated the unloading platform 36 to a vertical position, at which point the spring loaded hold-down mechanism 94 releases the slide carrier 60 into the output hopper 18. The carrier 60 is supported in the output hopper 18 by means of ears 64 (FIG. 5a and 5b). FIG. 7c shows the unloading platform 16 being rotated back towards the horizontal position. The platform 36 rotates upward and contacts the deposited carrier 60. The upward movement pushes the carrier toward the front of the output hopper 18. FIG. 7d shows the unloading platform 36 at its original horizontal position after having output a series of slide carriers 60 to the output hopper 18.

The aspects of the apparatus 10 relating to scanning, focusing and image processing are further described in U.S. Pat. No. 6,215,892, the disclosure of which is incorporated herein.

Aspects of the invention may be implemented in hardware or software, or a combination of both. However, preferably, the algorithms and processes of the invention are implemented in one or more computer programs executing on programmable computers each comprising at least one processor, at least one data storage system (including volatile and nonvolatile memory and/or storage elements), at least one input device, and at least one output device. Program code is applied to input data to perform the functions described herein and generate output information. The output information is applied to one or more output devices, in known fashion.

Each program may be implemented in any desired computer language (including machine, assembly, high level procedural, or object oriented programming languages) to communicate with a computer system. In any case, the language may be a compiled or interpreted language.

Each such computer program is preferably stored on a storage media or device (e.g., ROM, CD-ROM, tape, or magnetic diskette) readable by a general or special purpose programmable computer, for configuring and operating the computer when the storage media or device is read by the computer to perform the procedures described herein. The inventive system may also be considered to be implemented as a computer-readable storage medium, configured with a computer program, where the storage medium so configured causes a computer to operate in a specific and predefined manner to perform the functions described herein.

### EXAMPLES

20 ml of peripheral blood was drawn and anticoagulated with EDTA. The red blood cells were lysed for 5 minutes at room temperature with a red blood cell lysis buffer at a final concentration of 155 mM NH₄C1, 10 mM KHCO₃, 0.1 mM EDTA, at pH 7.2. Whole cells were separated from lysed red blood cells by centrifugation at 300 RCF for 5 minutes at room temperature. The supernatant was carefully aspirated and the RBC lysis step was repeated a second time with fresh lysis buffer. The supernatant was carefully aspirated again and the pellet was washed in PEB (PBS, EDTA, BSA; 1×PBS, 0.1 mM EDTA, and 0.5% BSA) with an additional 5 minutes centrifugation tube. A 5 μl aliquot of the cell suspension was kept separately in a microcentrifuge tube to be added later in the cytospin of the positively selected cells to provide a minimum amount of cells at the end of this procedure.

Carcinoma cells expressing the human Epithelial Antigen (recognized by the monoclonal antibody HAE125) were enriched with magnetic beads by adding 0.1 ml of HAE125-microbeads (Miltenyi Biotec) to the 0.9 ml of cell suspension in the 1.5 ml microcentrifuge tube and incubated for 30 minutes at room temperature on an orbital shaker or a rotisserie.

An LS+ column on a midiMACS magnet (Miltenyi Biotec) was mounted and prepared with 3 ml of PEB. The cell suspension was loaded on the column, followed by 2 ml of PEB and 4 ml of PBS. The flow through was collected in a tube for the negative selection step. The column was eluted by removing the midiMACS magnet from the column and placing the column over a large capacity cytospin chamber (Hettich #1666). 3 ml of PBS buffer was added to the column and collected in the chamber by gravity elution. A second 3 ml volume of PBS was added to the column and eluted by positive pressure (i.e., gently pushed through). The eluant was collected in the chamber and mixed with the 5 μl aliquot of cell suspension taken before the positive selection. The cells from the eluant and the aliquot were spun together onto a slide in a cytocentrifuge at 500 RPM with a Hettich Universal 16A centrifuge (RevPro) for 15 minutes at room temperature. The slides were removed and allowed to dry for at least 1 hour at room temperature. The cell/magnetic bead ratio of the negative selection step can be optimized by one skilled in the art. In addition, the total cell number should not exceed the capacity (e.g., 100 million cells) of the column used in the negative selection.

Based on the WBC titer determined in the sample of peripheral blood, the number of WBC originally present in the sample was calculated. Using this number, the volume of the flow through containing 100 million WBC was then determined and transferred to a separate tube and spun at 300 RCF for 5 minutes. The cells in the pellet were resuspended in 0.9 ml PEB and transferred in a microcentrifuge tube. A 5 μl aliquot of the cell suspension was kept with 0.5 ml PEB in the tube that was used to collect the flow through from the negative selection. The purpose of this aliquot was to provide a minimum of amount of cells at the end of the procedure.

A volume of 150 μl of CD45-microbeads (Miltenyi Biotec, Inc.) was added to the microfuge tube containing 0.9 ml of cell suspension and incubated for 15 minutes at room temperature on an orbital shaker or rotisserie. A LS+ column (Miltenyi Biotec, Inc.) was mounted and washed with 3 μl PEB. After the wash, the tube containing the 5 μl aliquot was placed below the column. At the end of the 15 minute microbead-cell incubation, 5 μl PEB was loaded on the column. The cell-microbead mixture was immediately added to the PEB on the top of the column. The microfuge tube that contained the cell suspension was washed with 1 ml PEB, which was added to the column. When the top of the column was empty, the column was washed with 5 ml PEB. The flow through was clear, indicating that the negative selection worked properly. The tube containing the flow through from the negative selection was spun at 300 RCF for 5 minutes. The cells from the pellet were resuspended in 1.5 ml PBS and spun onto slides at low speed (500 rpm=32 RCF) with a Hettich Universal 16A centrifuge for 15 minutes at room temperature.

After the cytospin, the slides were removed and allowed to dry for at least 1 hour at room temperature. The slides were fixed with 400 μl 0.5% formalin for 10 minutes at room temperature in a moist chamber and washed two times in PBS for 3 minutes each. Permeabilization was performed by using standard buffers in Coplin jars. Antibodies specifically recognizing cytokeratins were mixed and incubated with the slides for 45 minutes or more at room temperature. The slides were washed and stained with standard buffers and chromogens for 10 minutes at room temperature. The slides were washed once in PBS and once in deionized water. Counterstaining with hematoxylin (DAKO #S3309) was performed for 4 seconds in concentrated stock solution, or for 20 seconds in a 5× diluted solution. The slides were then washed, incubated 30 seconds in a blueing solution (NH4OH 0.08%), washed again in deionized water and dried at 70° C. The slides were finally mounted with a cellulose film (Sakura TissueTek SCA #4770) using xylene or, alternatively, mounted with Permount (Fisher #SP-15-100) and a 24×30 mm No. 1 glass coverslip (Fisher #125485G).

To enrich the carcinoma cells expressing the human Epithelial Antigen (recognized by the monoclonal antibody HAE125), 20 ml of peripheral blood was drawn and anticoagulated with EDTA. The red blood cells were lysed for 5 minutes at room temperature with a red blood cell lysis buffer at a final concentration of 155 mM NH₄C1, 10 mM KHCO₃, 0.1 mM EDTA at pH 7.2. Whole cells were separated from the lysed RBCs by centrifugation at 300 RCF for 10 minutes at room temperature. The supernatant was carefully aspirated and the pellet resuspended in PEB (PBS, EDTA, BSA; 1×PBS, 0.1 mM EDTA, and 0.5% BSA). The cell pellet was washed one time with an additional centrifugation and resuspension step as described above. The final pellet was resuspended in 0.9 ml of PEB and transferred to a 1.5 ml microcentrifuge tube.

Carcinoma cells were enriched with magnetic beads by adding 0.2 ml of HAE125-microbeads (Miltenyi Biotec Inc.) to the 0.9 ml of cell suspension in the 1.5 ml microcentrifuge tube and incubated for 30 minutes at room temperature on an orbital or rotary shaker.

An LS+ column on a midiMACS magnet (Miltenyi Biotec) was mounted and washed with 3 ml of PEB. The cell suspension was loaded on the column followed by 2 ml of PEB and 4 ml of PBS. The column was eluted by removing the midiMACS magnet from the column and placing the column over a Hettich #1666 chamber. 3 ml of PBS buffer was added to the column and collected in the chamber by gravity elution. A second 3 ml volume was added to the column and eluted by positive pressure (i.e., gently pushed through). The eluant was collected in the chamber and spun onto slides in a cytocentrifuge at 500 RPM with a Hettich 16A centrifuge (RevPro) for 15 minutes at room temperature.

The slides were removed and allowed to dry for at least 1 hour at room temperature. The slides were fixed in 400 μl 0.5% formalin for 10 minutes at room temperature in a moist chamber and washed 2× in PBS for 3 minutes each. Permeabilization was performed by using standard buffers in Coplin Jars. Antibodies were mixed and incubated with the slides for 45 minutes at room temperature. The slides were washed and stained with standard buffers for 10 to 15 minutes at room temperature. The slides were washed once in PBS and once in deionized water. Counterstaining with Mayer's hematoxylin (DAKO #S3309) was performed for 4 seconds in concentrated stock solution or 20 seconds in a 5× diluted solution. The slides were then washed, dried for 20 minutes at 70° C., and mounted with an automated coverslipper (Sakura Tissue-Tek SCA), or manually with a glass coverslip and standard mounting medium, such as permount (Fisher #SP-15-100).

The mounted and fixed slides comprising the enriched sample are then loaded on to the automated analysis system as described above, and processed to identify “rare events”.

The following protocol is designed to enrich for carcinoma cells expressing the human Epithelial Antigen (recognized by the monoclonal antibody HAE125), starting with 20 ml of peripheral blood containing EDTA. In order to keep a precise schedule, it is recommended not to use more than three samples simultaneously.

Red Blood Cells Lysis: A 1× lysis buffer is prepared from a 10× stock solution with deionized or distilled water. 80 ml 1× lysis buffer is used for each 20 ml blood sample. 20 ml fresh blood is equally split in 2 disposable 50 ml conical tubes (label tubes). In each conical tube, 40 ml 1× lysis buffer is added and mixed by inverting the tubes; the tubes are kept at room temperature for 5 minutes. The tubes are then spun at 300 RCF for 10 minutes at room temperature. The supernatant is carefully removed by aspiration. Each pellet is resuspended gently in 5 ml PEB by pipetting 3-5× and swirling. The cells are pooled together into one conical tube. Each empty tube is washed one time and the wash is combined with the pooled cells. The tube is respun at 300 RCF for 5 minutes at room temperature and the aspirate is carefully removed. The cells are gently resuspended in 0.9 ml PEB total as follows: the pellet's volume is ˜200 μl; add first 500 μl PEB with a P1000 Pipetman to the pellet; (iii) resuspend the cells very gently by pipetting and swirling the cells; (iv) transfer the cells into a 1.5 ml Eppendorf tube; and (v) rinse the bottom of the 50 ml conical tube with 200 μl PEB and add this to the Eppendorf tube (do not use small (P200) conical tips with the cells). 0.1 ml HAE125 microbeads (Miltenyi Biotec Inc.) is added to the 0.9 ml of cell suspension in the 1.5 ml Eppendorf tube. The mixture is incubated 30 minutes at room temperature on an orbital shaker or a rotisserie (such as the “Labquake” tube rotator from Barnstead/Thermolyne). The LS+ column is assembled on a MidiMACS magnet (Miltenyi Biotec), mounted on the black metallic rack. Make sure that the little wings of the column are in the front. Wash the LS+ column mounted on a MidiMACS magnet with 3 ml PEB. Put a 15 ml conical tube below the column to collect the flow through. Load the cells on the column. Wash the Eppendorf tube with 0.5 ml PEB and add it on the column. Wash the loaded column as follows: (i) add 2 ml PEB and let it go through; (ii) add 2 ml PBS; and (iii) add 2 ml PBS again. A DAKO silanized slide is assembled with a carrier (Hettich #1670), a large chamber (Hettich #1666), and the corresponding ring for cytospin. Prepare a second chamber, if it is necessary to have a balancer during the cytospin, and fill it with 6 ml of water or PBS.

Remove the column from the magnet and install it on a clip of the rotary shaker used as a rack (put the Hettich chamber below the column). Add 3 ml PBS buffer on the column and let it drip by gravity into the assembled cytospin chamber. Repeat this with 3 ml PBS and, in this case, push the buffer gently through the column with the corresponding plunger and collect it in the cytospin chamber (do not blow air through the column by pushing the plunger too far).

The cells are cytospun at the lowest speed (˜250 RMP) with a Hettich 16A centrifuge (RevPro) for 15 minutes at room temperature. Eliminate the supernatant with a vacuum pump linked to a Pasteur pipet having a disposable conical tip at its end. Disassemble the chamber and dry the slide for at least 1 hour at room temperature. The slide is ready to be stained for cytokeratin.

After cytospin, the slide is dried for about 1 hour at room temperature. The spot containing the cytospun cells is circled with a hydrophobic pen (such as DAKO Pen S2002), keeping the gap of ˜5 mm in between.

A 20× stock solution: 10% formalin, neutralized (SIGMA #HT50-1-128) is diluted to 1× in PBS (0.5% formalin) just prior to use. 400 μl 0.5% formalin is incubated on the spot for 10 minutes at room temperature in a moist chamber. 2× PBS is used for washing (3 minutes each wash). The sample dot is permeabilized for 5 minutes at room temperature in a Coplin Jar. After permeablization the slide is washed 3× in PBS for 3 minutes each. After washing the slide is incubated with the primary antibody. The sample is counterstained with Mayer's hematoxylin (DAKO #S3309) for 4 seconds in concentrated stock solution or 20 seconds in a 5× diluted (+dH20) solution. The slide is then washed in 1× H₂O, up/down 10× in a beaker, then 2× deionized H₂O for 3 minutes in a Coplin Jar. The slides are then dried for 20 minutes at 70° C. and mounted with a glass coverslip 24×30 mm No. 1 (such as Fisher #125485G). The coverslip is kept away from the last 5 mm of the bottom of the slide to avoid the coverslip being squeezed by the carrier used in the automated imaging system. The coverslip is gently pressed to eliminate the surplus of mounting medium and to minimize the distance between the coverslip and the slide. This medium is permanent and does not need additional sealing. As an alternative to a glass coverslip, the sample can be covered with a cellulose film from SAKURA Tissue-Tek automated coverslipper, using xylene. This method is permanent and does not introduce bubbles.

A number of embodiments of the invention have been described. Nevertheless, it will be understood that various modifications may be made without departing from the spirit and scope of the invention. Accordingly, other embodiments are within the scope of the following claims.

What is claimed is:

1. A method for identifying rare events in a biological sample, comprising:
obtaining a source of cells;
contacting the source with a binding agent specific for a cell specific marker associated with a rare event wherein the binding agent is bound to a magnetic bead and wherein the binding agent binds to cells in the source expressing the cell specific marker;
separating cells bound by the binding agent from the source thereby obtaining a sub-population of cells enriched for the cell specific marker associated with the rare event;
placing the enriched sample on a substrate;
automatically scanning the substrate at a plurality of coordinates;
automatically obtaining a plurality of images at locations on the substrate that comprise the enriched sample; and
processing the plurality of image to identify the rare event.

2. The method according to claim 1, wherein the binding agent is an antibody.

3. The method according to claim 1, wherein the sub-population is enriched for carcinoma cells.

4. The method of claim 1, wherein the separating is done by positive selection.

5. The method of claim 1, wherein the separating is done by negative selection.

6. The method of claim 2, wherein the antibody is monoclonal or polyclonal.

7. The method of claim 2, wherein the antibody recognizes an epithelial marker.

8. The method of claim 2, wherein the antibody is selected to avoid cross reactivity with the beads.

9. The method of claim 3, wherein the carcinoma cells are from peripheral blood.

10. The method of claim 1, further comprising:
(a) automatically identifying a coordinate of the rare event; and
(b) automatically acquiring an image of the rare event, at the location coordinates.

11. The method of claim 1, wherein the rare event is detected by immunohistochemistry.

12. The method of claim 1, wherein the rare event is detected by in situ hybridization.

13. The method of claim 1, wherein the rare event is detected by a stain.

14. The method of claim 13, wherein the stain is a nucleic acid dye selected from the group consisting of hematoxylin, Giemsa stain, methyl green, Nuclear Fast-Red, Hoechst 33342, Hoechst 33258, thiazole orange, DAPI, ethidium bromide, propidium iodide, TOTO, YOYO-1, SYTOX Blue, SYTOX Green, 7-Aminoactinomycin, 9-Amino-6-chloro-2-methoxyacridine, and acridine homodimer.

15. The method of claim 13, wherein the rare event is stained with a cytoplasmic dye such as eosin or Kleihauer-Betke cytochemical stain or a combination thereof.

16. The method of claim 1, wherein the cell specific marker is detected by a nuclear stain and counterstain.

17. The method of claim 1, wherein the cell specific marker is detected by immunohistochemistry, in situ hybridization, staining or a combination thereof.

18. The method of claim 1, wherein the image is a digital image.
